(19) United States
(12) Patent Application Publication
(10) Pub. No.: US 2022/0331228 A1
(43) Pub. Date: Oct. 20, 2022

(54) ANHYDROUS COMPOSITION COMPRISING AT LEAST ONE AMINO SILICONE, AT LEAST ONE ALKOXYSILANE AND AT LEAST ONE COLORING AGENT

(71) Applicant: L'OREAL, Paris (FR)

(72) Inventors: Alexis LIARD, SAINT-OUEN (FR); Marine PROTAT-GERARDIN, SAINT-OUEN (FR)

(21) Appl. No.: 17/620,946

(22) PCT Filed: Jun. 17, 2020
(86) PCT No.: PCT/EP2020/066786

§ 371 Date: Dec. 20, 2021

(30) Foreign Application Priority Data
Jun. 24, 2019 (FR) FR1906827

(51) Int. Cl. A61K 8/898; A61K 8/58; A61Q 1/14; A61Q 5/06

(52) U.S. Cl. CPC A61K 8/898; A61K 8/585; A61Q 1/14; A61Q 5/065; A61K 2800/43; A61K 2800/31

(57) ABSTRACT

The present invention relates to an anhydrous composition comprising at least one amino silicone, at least one alkoxysilane of formula (VIII), and at least one coloring agent chosen from pigments, direct dyes and mixtures thereof. The present invention also relates to a process for treating keratin fibers such as the hair.

The present invention relates to an anhydrous composition comprising at least one amino silicone, at least one alkoxysilane of formula (VIII) as described below, and at least one coloring agent chosen from pigments, direct dyes and mixtures thereof.

The present invention also relates to a process for treating keratin fibers such as the hair, comprising the application of a dye composition to the keratin fibers.

### TECHNICAL FIELD

In the field of dyeing keratin fibers, in particular human keratin fibers, it is already known practice to dye keratin fibers via various techniques using direct dyes or pigments for nonpermanent dyeing, or dye precursors for permanent dyeing.

There are essentially three types of process for dyeing the hair:

a) “permanent” dyeing, the function of which is to afford a substantial modification to the natural color and which uses oxidation dyes which penetrate into the hair fiber and forms the dye via an oxidative condensation process;

b) nonpermanent, semipermanent or direct dyeing, which does not use the oxidative condensation process and withstands four or five shampoo washes; it consists in dyeing keratin fibers with dye compositions containing direct dyes.

c) temporary dyeing, which gives rise to a modification of the natural color of the hair that remains from one shampoo washing to the next, and which serves to enhance or correct a shade that has already been obtained. It may also be likened to a “makeup” process.

For this last type of dyeing, it is known practice to use colored polymers formed by grafting one or more dyes of azo, triphenylmethane, azine, indoamine or anthraquinone nature onto a polymer chain. These colored polymers are not entirely satisfactory, notably as regards the homogeneity of the coloring obtained and its resistance, not to mention the problems associated with their manufacture and notably with their reproducibility.

Another dyeing method consists in using pigments. Specifically, the use of pigment on the surface of keratin fibers generally makes it possible to obtain colorings that are visible on dark hair, since the surface pigment masks the natural color of the fiber. However, the colorings obtained via this dyeing method have the drawback of having poor resistance to shampoo washing and also to external agents such as sebum, perspiration, brushing and/or rubbing.

In addition, compositions for temporarily dyeing the hair may also lead to a hair feel that is not natural and/or is not cosmetic; the hair thus dyed may notably lack softness and/or suppleness and/or strand separation.

In addition, there are no effective makeup-removing compositions for removing this type of temporary dye composition when it is persistent with respect to shampoo washing.

The need thus remains for a composition for treating keratin fibers, notably the hair, which has the advantage of obtaining a homogeneous and smooth colored coating on the hair, and also hair with complete strand separation, while at the same time forming a coating that is persistent with respect to shampoo washing and to the various attacking factors to which the hair may be subjected, such as brushing and/or rubbing, without degrading the hair. There is also a need to be able to eliminate this colored coating when so desired.

Thus, the aim of the present invention is to develop a composition for treating keratin fibers, notably the hair, which has the advantage of obtaining a homogeneous and smooth colored coating on the hair, and also hair with complete strand separation, while at the same time forming a coating that is persistent with respect to shampoo washing and to the various attacking factors to which the hair may be subjected, such as brushing and/or rubbing, without degrading the hair. Advantageously, the colored coating can be readily eliminated when so desired.

### DISCLOSURE OF THE INVENTION

One subject of the present invention is thus an anhydrous composition comprising:

a) at least one amino silicone,
b) at least one alkoxysilane chosen from the compounds of formula (VIII) as described below, oligomers thereof and/or mixtures thereof, and
c) at least one coloring agent chosen from pigments, direct dyes and mixtures thereof.

The present invention also relates to a cosmetic process for treating, in particular dyeing, human keratin fibers such as the hair, in which the composition as described below is applied to said fibers.

The present invention also relates to the use of the composition as described below for the cosmetic treatment of, in particular for dyeing, keratin fibers, in particular human keratin fibers such as the hair.

Through the use of this composition, colored coatings are obtained on the hair that make it possible to obtain a coloring that is visible on all types of hair in a manner that is persistent with respect to shampoo washing, while at the same time preserving the physical qualities of the keratin fibers. Such a coating may be resistant to the external attacking factors to which the hair may be subjected, such as blow-drying and perspiration. It makes it possible in particular to obtain a smooth and uniform deposit.

Moreover, this composition makes it possible to obtain hair with complete strand separation, which can be styled without any problem.

The term “hair with strand separation” means hair which, after application of the composition and drying, is not stuck together (or of which all the strands are separated from each other) and thus does not form clumps of hair.

For the purposes of the present invention, the term “coloring that is persistent with respect to shampoo washing” means that the coloring obtained persists after one shampoo wash, preferably after three shampoo washes, more preferentially after five shampoo washes.

Advantageously, the colored coating thus obtained can be readily eliminated by means of a makeup-removing composition.

The term “at least one” means “one or more”.

The invention is not limited to the illustrated examples. The features of the various examples may notably be combined within variants which are not illustrated.

For the purposes of the present invention and unless otherwise indicated:
an “alkyl” radical denotes a linear or branched saturated radical containing, for example, from 1 to 20 carbon atoms;
an “aminoalkyl” radical denotes an alkyl radical as defined previously, said alkyl radical comprising an NH₂ group;
a “hydroxyalkyl” radical denotes an alkyl radical as defined previously, said alkyl radical comprising an OH group;
an “alkylene” radical denotes a linear or branched divalent saturated C₂-C₄ hydrocarbon-based group such as methylene, ethylene or propylene;
a “cycloalkyl” radical denotes a cyclic saturated hydrocarbon-based group comprising from 1 to 3 rings, preferably 2 rings, and comprising from 3 to 20 carbon atoms, preferably between 5 and 10 carbon atoms, such as cyclopentyl, cyclohexyl, cycloheptyl, norbornyl, or isobornyl, the cycloalkyl radical possibly being substituted with one or more (C₁-C₄)alkyl groups such as methyl; preferably, the cycloalkyl radical is an isobornyl group;
an “aryl” radical is a cyclic unsaturated aromatic radical comprising from 6 to 12 carbon atoms, which is mono- or bicyclic, fused or unfused; preferably, the aryl group comprises 1 ring containing 6 carbon atoms, such as phenyl;
an “acyloxy” radical denotes an aryl-oxy radical with “aryl” as defined previously;
an “alkoxy” radical denotes an alkyl-oxy radical with “alkyl” as defined previously;
an “acyloxy” radical denotes a radical R—COO with R being an alkyl group as defined previously.

The anhydrous composition according to the invention is preferably an anhydrous composition for treating human keratin fibers such as the hair, more preferentially an anhydrous composition for dyeing human keratin fibers such as the hair.

The term “keratin fibers” particularly means human keratin fibers such as head hair, eyelashes, eyebrows, and bodily hair, preferentially head hair, eyebrows and eyelashes, even more preferentially head hair.

### Anhydrous Composition

The composition according to the present invention is an anhydrous composition.

The term “anhydrous composition” means a composition with a water content of less than 3% by weight, preferably less than 2% by weight and even more preferably less than 1% by weight relative to the total weight of the composition.

More preferentially, the composition is free of water (zero content). The water is not added during the preparation of the composition, but may correspond to the residual water provided by the mixed ingredients.

### Amino Silicone:

The composition according to the invention comprises at least one amino silicone.

The term “amino silicone” denotes any silicone including at least one primary, secondary or tertiary amine function or a quaternary ammonium group.

The weight-average molecular masses of these amino silicones may be measured by gel permeation chromatography (GPC) at room temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

The amino silicone that may be used in the context of the invention may be chosen from the compounds of formula (I) below

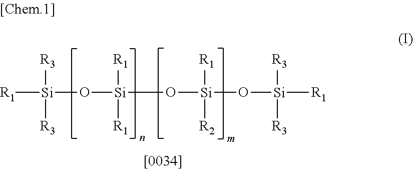

in which:
R₁, which may be identical or different, represents a hydrogen atom, a phenyl group, a hydroxyl group, a C₁-C₈ alkyl group, for example a methyl, or a C₁-C₆ alkoxy group, for example methoxy;
R₂ represents a monovalent radical of formula —CqH2qL in which q is a number ranging from 2 to 8 and L is an optionally quaternized amino group chosen from the following groups:

—N(R″)₂;—N+(R″)₃A⁻;—NR″-Q-N(R″)₂ and —NR″-Q-N⁺(R″)₃A⁻,

in which R″, which may be identical or different, represents a hydrogen atom, a phenyl group, a benzyl group or a saturated monovalent hydrocarbon-based radical, for example a C₁-C₂₀ alkyl group; Q denotes a linear or branched group of formula CrH₂r, r being an integer ranging from 2 to 6, preferably from 2 to 4; and A⁻ represents a cosmetically acceptable anion, notably a halide such as fluoride, chloride, bromide or iodide;
R₃, which may be identical or different, represents a C₁-C₈ alkyl group, for example a methyl, or a monovalent radical of formula —CqH₂qL in which q is a number ranging from 2 to 8 and L is an optionally quaternized amine group chosen from the following groups:

—N(R″)₂;—N+(R″)₃A⁻;—NR″-Q-N(R″)₂ and —NR″-Q-N⁺(R″)₃A⁻,

in which R″, which may be identical or different, represents a hydrogen atom, a phenyl group, a benzyl group or a saturated monovalent hydrocarbon-based radical, for example a C₁-C₂₀ alkyl group; Q denotes a linear or branched group of formula CrH₂r, r being an integer ranging from 2 to 6, preferably from 2 to 4; and A⁻ represents a cosmetically acceptable anion, notably a halide such as fluoride, chloride, bromide or iodide; and
m and n are numbers such that the sum (n+m) ranges from 1 to 2000, in particular from 50 to 150, m possibly denoting a number from 0 to 1999 and notably from 49 to 149, and n possibly denoting a number from 1 to 2000 and notably from 1 to 10.

Preferably, R₁, which may be identical or different, represents a hydroxyl group or a C₁-C₆ alkoxy group, for example methoxy;
R₂ represents a monovalent radical of formula —CqH₂qL in which q is a number ranging from 2 to 8 and L is an amine group chosen from the following groups:

—N(R₄)₂;

—N(R₄)—CH₂—CH₂—N(R₄)₂;

in which R₁ represents a hydrogen atom; a phenyl group; a benzyl group or a saturated monovalent hydrocarbon-based radical, for example a C₁-C₂₀ alkyl group;
R₃, which may be identical or different, represents a C₁-C₈ alkyl group, for example a methyl; and
m and n are numbers such that the sum (n+m) ranges from 1 to 2000, in particular from 50 to 150, m possibly denoting a number from 0 to 1999 and notably from 49 to 149, and n possibly denoting a number from 1 to 2000 and notably from 1 to 10.

According to another embodiment, the amino silicone(s) correspond to polysiloxanes corresponding to formula (A) below:

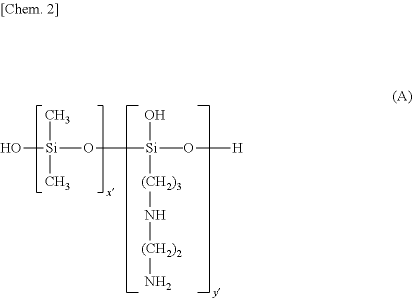

in which x′ and y′ are integers such that the weight-average molecular weight (Mw) is of between 5000 and 500 000 approximately;

According to a preferred embodiment, the amino silicone(s) are chosen from the silicones of formula (II) below:

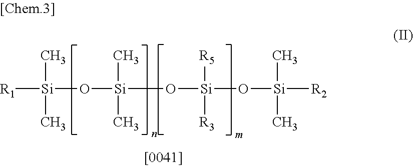

in which:
m and n are numbers such that the sum (m+n) ranges from 1 to 1000, in particular from 50 to 350 and more particularly from 150 to 250, n possibly denoting a number between 0 and 999, in particular from 49 to 349 and more particularly from 159 to 239, and m possibly denoting a number from 1 to 1000, in particular from 1 to 50;
R₁ and R₂, which may be identical or different, represent a C₁-C₈ alkyl group, for example a methyl, a hydroxyl group or a C₁ to C₆ and preferably C₁ to C₄ alkoxy group;
R₃ represents a monovalent radical of formula —CqH2qL in which q is a number ranging from 2 to 8 and L is an amino group chosen from the following groups:

—N(R₄)₂;

—N(R₄)—CH₂—CH₂—N(R₄)₂;

in which R₁ represents a hydrogen atom; a phenyl group; a benzyl group or a saturated monovalent hydrocarbon-based radical, for example a C₁-C₂₀ alkyl group; and
R₅ represents a hydroxyl group, a C₁-C₈ alkyl group, for example a methyl, or a C₁-C₆ alkoxy group, for example methoxy.

More preferentially, the amino silicone(s) are chosen from the silicones of formula (III) below:

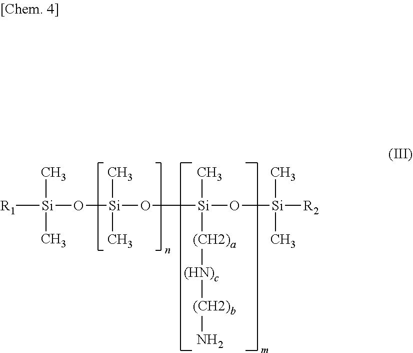

in which:
m and n are numbers such that the sum (m+n) ranges from 1 to 1000, in particular from 50 to 350 and more particularly from 150 to 250, n possibly denoting a number between 0 and 999, in particular from 49 to 349 and more particularly from 159 to 239, and m possibly denoting a number from 1 to 1000, in particular from 1 to 50,
R₁ and R₂, which may be identical or different, represent a C₁ to C₄ alkoxy group, preferably a methoxy radical,
a denotes an integer ranging from 0 to 6,
b denotes an integer ranging from 1 to 6,
c denotes an integer equal to 0 or 1.

Preferably, R₁ and R₂ are identical.
from the silicones of formula (IV) below:

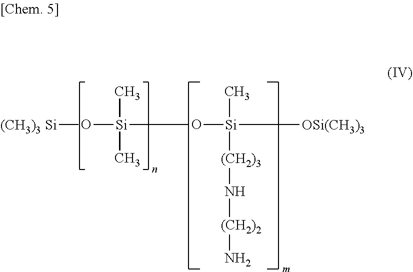

in which m and n are numbers such that the sum (n+m) ranges from 1 to 2000, in particular from 50 to 150, n possibly denoting a number from 0 to 1999 and notably from 49 to 149, and m possibly denoting a number from 1 to 2000 and notably from 1 to 10.
from the silicones of formula (V) below:

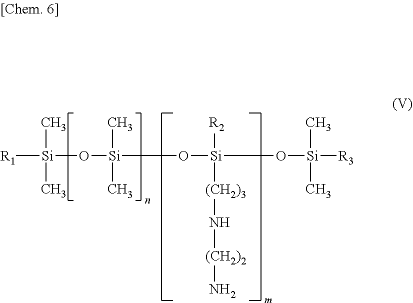

in which:
m and n are numbers such that the sum (n+m) ranges from 1 to 1000, in particular from 50 to 250 and more particularly from 100 to 200; n possibly denoting a number from 0 to 999 and notably from 49 to 249 and more particularly from 125 to 175, and m possibly denoting a number from 1 to 1000, notably from 1 to 10, more particularly from 1 to 5;
R₁, R₂ and R₃, which may be identical or different, represent a hydroxyl or a C₁-C₄ alkoxy radical, at least one of the radicals R₁ to R₃ denoting an alkoxy radical.

The hydroxy/alkoxy mole ratio preferably ranges from 0.2:1 to 0.4:1 and preferably from 0.25:1 to 0.35:1 and is more particularly equal to 0.3:1.

The weight-average molecular mass (Mw) of these silicones of formula (V) preferably ranges from 2000 to 1 000 000, more particularly from 3500 to 200 000.

A product containing amino silicones of structure (V) is sold by the company Wacker under the name Belsil® ADM 652.
from the silicones of formula (VI) below:

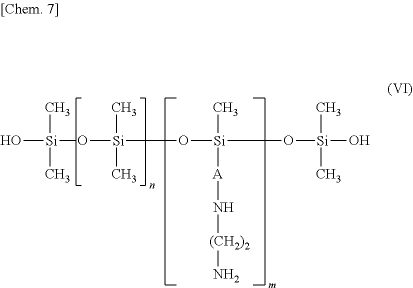

in which:
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
A denotes a linear or branched alkylene radical containing from 4 to 8 carbon atoms and preferably 4 carbon atoms. This radical is preferably linear.

The weight-average molecular mass (Mw) of these amino silicones of formula (VI) preferably ranges from 2000 to 1 000 000 and even more particularly from 3500 to 200 000.

A silicone corresponding to this formula (VI) is, for example, Xiameter MEM 8299 Emulsion from Dow Corning.
from the silicones of formula (VII) below:

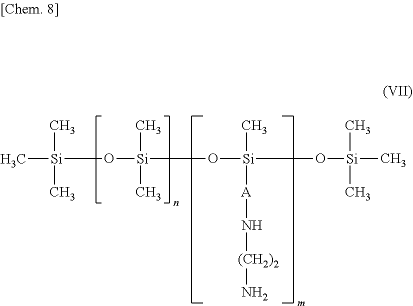

in which:
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
A denotes a linear or branched alkylene radical containing from 4 to 8 carbon atoms and preferably 4 carbon atoms. This radical is preferably branched.

The weight-average molecular mass (Mw) of these amino silicones of formula (VII) preferably ranges from 500 to 1 000 000 and even more particularly from 1000 to 200 000.

A silicone corresponding to this formula (VII) is, for example, DC2-8566 Amino Fluid from Dow Corning.
and mixtures thereof.

Even more preferentially, the amino silicone(s) are chosen from the silicones of formula (III).

The weight-average molecular mass (Mw) of an amino silicone of formula (III) preferably ranges from 1000 to 200 000 and even more particularly from 2000 to 100 000 and more particularly from 2000 to 50 000.

An amino silicone corresponding to formula (II) is, for example, the compound having the trade name KF 857, sold by the company Shin-Etsu, or the compound having the trade name KF 862, sold by the company Shin-Etsu.

The composition according to the invention may comprise one or more amino silicones present in a total amount ranging from 0.1% to 40% by weight, preferably from 0.5% to 30% by weight, preferentially from 1% to 20% by weight and better still from 5% to 15% by weight relative to the total weight of the composition.

### Alkoxysilane:

The composition according to the invention comprises at least one alkoxysilane chosen from the compounds of formula (VIII) below, oligomers thereof and/or mixtures thereof:

[Chem. 9]

R¹ₓSi(OR²)₍₄₋ₓ₎ (VIII)

in which:
R¹ represents an alkoxy group containing from 1 to 10 carbon atoms, a linear or branched, saturated or unsaturated, cyclic or acyclic C₁ to C₂₂ and notably C₁ to C₂₀ hydrocarbon-based radical, which may be substituted with at least one group chosen from a hydroxyl group (OH); a thiol group; an amino group NH₂; an alkylamino group NHR in which R denotes a linear or branched alkyl radical containing from 1 to 20 carbon atoms, notably from 1 to 10 carbon atoms, or an alkoxy group containing from 1 to 10 carbon atoms; a cycloalkyl containing from 3 to 40 carbon atoms; an aryl containing from 6 to 30 carbon atoms; R¹ possibly being interrupted with at least one heteroatom chosen from O, S, NH or a carbonyl group (CO);
R² represents a hydrogen atom or an alkyl group containing from 1 to 20 carbon atoms, preferably from 2 to 6 carbon atoms;
x denotes an integer ranging from 1 to 3.

The term “oligomer” means compound(s) including at least two silicon atoms obtained by oligomerization or polymerization of the compounds of formula (VIII).

Preferably, R¹ represents an alkoxy group containing from 1 to 10 carbon atoms, such as an ethoxy, or R¹ represents a linear or branched, saturated C₁ to C₂₂ and notably C₁ to C₂₀ hydrocarbon-based radical, which is preferably linear, said hydrocarbon-based radical possibly being substituted with at least one amino group NH₂ or an alkylamino group NHR, in which R denotes a linear or branched C₁ to C₂₀ and notably C₁ to C₁₀ alkyl.

More preferentially, R¹ represents a saturated linear C₁ to C₆ hydrocarbon-based radical, which may be substituted with an amino group NH₂.

Preferably, R² represents an alkyl group containing from 1 to 10 carbon atoms, preferably from 2 to 6 carbon atoms, more preferentially an ethyl group.

The alkoxysilane(s) of formula (VIII) may be chosen from the compounds of formula (VIIIa) and/or (VIIIb) below, alone or as a mixture:

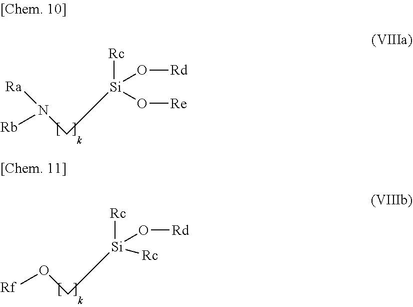

in which:
Ra and Rb, which may be identical or different, represent a hydrogen atom; an alkyl group containing from 1 to 20 carbon atoms, preferably from 1 to 10 carbon atoms; a cycloalkyl group containing from 3 to 20 carbon atoms, an aryl group containing from 6 to 12 carbon atoms; an aminoalkyl group containing from 1 to 20 carbon atoms; a hydroxyalkyl group containing from 1 to 20 carbon atoms; an alkoxy group containing from 1 to 10 carbon atoms; an alkylcarbonyl group containing from 1 to 10 carbon atoms, such as methylcarbonyl (i.e. acetyl);
Rc represents a hydrogen atom, an alkyl group containing from 1 to 20 carbon atoms, preferably a methyl, or an alkoxy group containing from 1 to 10 carbon atoms, preferably an ethoxy group; an alkylaryl group containing from 7 to 12 carbon atoms;
Rd and Re, which may be identical or different, represent an alkyl group containing from 1 to 20 carbon atoms, preferably an ethyl;
k denotes an integer ranging from 0 to 20, preferably ranging from 0 to 3;
Rf represents a hydrogen atom; an alkyl group containing from 1 to 20 carbon atoms such as an ethyl group or a group of formula (IX) below:

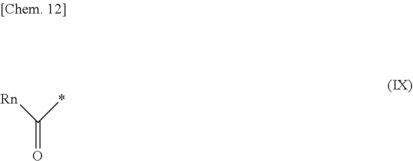

in which Rn represents a hydroxyl group or an alkyl group containing from 1 to 20 carbon atoms, preferably a methyl.

Among the alkoxysilanes of formula (VIIIa), the oligomers thereof and/or mixtures thereof, mention may notably be made of 3-aminopropyltriethoxysilane (APTES), 3-aminopropylmethyldiethoxysilane (APMDES) and N-cyclohexylaminomethyltriethoxysilane.

APTES may, for example, be sold by the company Dow Corning under the name Xiameter OFS-6011 Silane or under the name Silsoft A-1100 by the company Momentive Performance Materials.

The compounds of formula (VIIIa) may also denote Dynasylan SIVO 210 sold by the company Evonik.

N-cyclohexylaminomethyltriethoxysilane may be sold, for example, by the company Wacker under the name Geniosil XL 926.

Among the alkoxysilanes of formula (VIIIb), the oligomers thereof and/or mixtures thereof, mention may notably be made of tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof.

TEOS may be sold, for example, by the company Evonik under the name Dynasylan® A or Dynasylan® A SQ. MTES may be sold, for example, by the company Evonik under the trade name Dynasylan® MTES Diethyldiethoxysilane. DMDES may be sold, for example, by the company Gelest under the trade name SID3404.0.

Preferably, the alkoxysilane(s) chosen from the compounds of formula (VIII) are chosen from 3-aminopropyltriethoxysilane (APTES), 3-aminopropylmethyldiethoxysilane (APMDES), N-cyclohexylaminomethyltriethoxysilane, tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof, more preferentially 3-aminopropyltriethoxysilane (APTES), tetraethoxysilane (TEOS) and mixtures thereof.

According to a preferred embodiment, the alkoxysilane(s) of formula (VIII), oligomers thereof and/or mixtures thereof are chosen from the compounds of formula (VIIIa) below:

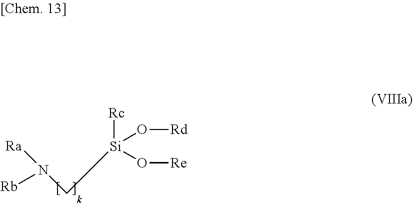

in which:
Ra and Rb, which are identical, represent a hydrogen atom;
Rc represents an alkyl group containing from 1 to 20 carbon atoms, preferably a methyl, or an alkoxy group containing from 1 to 10 carbon atoms, preferably an ethoxy group;
Rd and Re, which may be identical or different, represent an alkyl group containing from 1 to 20 carbon atoms, preferably an ethyl;
k denotes an integer ranging from 0 to 20, preferably ranging from 0 to 3.

Preferentially, Ra and Rb represent a hydrogen atom, Rc represents an ethoxy group, Rd and Re are identical and represent an ethyl and k is equal to 3.

More preferentially, the alkoxysilane of formula (VIII) is 3-aminopropyltriethoxysilane (APTES).

The composition according to the invention may comprise one or more alkoxysilanes of formula (VIII) present in a total amount ranging from 0.1% to 30% by weight, preferably from 0.2% to 20% by weight and better still from 0.5% to 10% by weight relative to the total weight of the composition.

Pigments and/or Direct Dyes:

The composition according to the invention comprises at least one coloring agent chosen from pigments, direct dyes and mixtures thereof.

Preferably, the composition according to the invention comprises one or more pigments.

The term “pigment” means any pigment that gives color to keratin materials. Their solubility in water at 25° C. and at atmospheric pressure (760 mmHg) is less than 0.05% by weight, and preferably less than 0.01%.

The pigments that may be used are notably chosen from the organic and/or mineral pigments known in the art, notably those described in Kirk-Othmer's Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” means any pigment that satisfies the definition in Ullmann's encyclopedia in the chapter on inorganic pigments. Among the mineral pigments that are useful in the present invention, mention may be made of ochres such as red ochre (clay (in particular kaolinite) and iron hydroxide (for example hematite)), brown ochre (clay (in particular kaolinite) and limonite), yellow ochre (clay (in particular kaolinite) and goethite); titanium dioxide, optionally surface-treated; zirconium oxide or cerium oxide; zinc oxide, iron oxide (black, yellow or red) or chromium oxide; manganese violet, ultramarine blue, chromium hydrate and ferric blue; metal powders such as aluminum powder or copper powder.

Mention may also be made of carbonates of alkaline-earth metals (such as calcium or magnesium), silicon dioxide, quartz, and also any other compound used as inert filler in cosmetic compositions, provided that these compounds afford the composition color or whiteness under the conditions in which they are used.

The pigment may be an organic pigment. The term “organic pigment” means any pigment that satisfies the definition in Ullmann's encyclopedia in the chapter on organic pigments.

The organic pigment may notably be chosen from nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

Use may also be made of any mineral or organic compound that is insoluble in the composition and that is conventional in the cosmetics field, provided that these compounds afford the composition color or whiteness under the conditions in which they are used, for example guanine, which, according to the refractive index of the composition, is a pigment.

In particular, the white or colored organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow IOG: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including a mineral core, at least one binder, for attaching the organic pigments to the core, and at least one organic pigment which at least partially covers the core.

The organic pigment may also be a lake. The term “lake” means dyes adsorbed onto insoluble particles, the assembly thus obtained remaining insoluble during use.

The inorganic substrates onto which the dyes are adsorbed are, for example, alumina, silica, calcium sodium borosilicate, calcium aluminum borosilicate and aluminum.

Among the dyes, mention may be made of carminic acid. Mention may also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a colored appearance (characterized by a certain shade, a certain vivacity and a certain level of luminance) that is nonuniform and that changes as a function of the conditions of observation (light, temperature, angles of observation, etc.). They thereby differ from colored pigments that afford a standard uniform opaque, semitransparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica coated with titanium or with bismuth oxychloride, colored nacreous pigments such as mica coated with titanium and with iron oxides, mica coated with iron oxide, mica coated with titanium and notably with ferric blue or with chromium oxide, mica coated with titanium and with an organic pigment as defined previously, and also nacreous pigments based on bismuth oxychloride. Nacreous pigments that may be mentioned include the Cellini nacres sold by BASF (mica-TiO2-lake), Prestige Bronze sold by Eckart (mica-Fe2O3), and Colorona sold by Merck (mica-TiO2-Fe2O3).

Mention may also be made of the gold-colored nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC1080RY by the company Toyal.

Finally, examples of nacres that may also be mentioned include polyethylene terephthalate flakes, notably those sold by the company Meadowbrook Inventions under the name Silver 1P 0.004X0.004 (silver flakes). Multilayer pigments based on synthetic substrates such as alumina, silica, sodium calcium borosilicate or calcium aluminum borosilicate, and aluminum, may also be envisaged.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. more luminous points that contrast with their environment by appearing to sparkle.

The reflective particles may be selected so as not to significantly alter the coloring effect generated by the coloring agents with which they are combined, and more particularly so as to optimize this effect in terms of color rendition. They may more particularly have a yellow, pink, red, bronze, orange, brown, gold and/or coppery color or tint.

These particles may have varied forms and may notably be in platelet or globular form, in particular in spherical form.

Irrespective of their form, the reflective particles may or may not have a multilayer structure, and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A-10158541, JP-A-07258460 and JP-A-05017710.

Still as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminum, iron, chromium, nickel, molybdenum, gold, copper, zinc, tin, magnesium, steel, bronze or titanium, said substrate being coated with at least one layer of at least one metal oxide, such as titanium oxide, aluminum oxide, iron oxide, cerium oxide, chromium oxide, silicon oxides and mixtures thereof.

Examples that may be mentioned include aluminum powder, bronze powder or copper powder coated with SiO₂ sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductor nanoparticles that are capable of emitting, under light excitation, radiation with a wavelength of between 400 nm and 700 nm. These nanoparticles are known from the literature. In particular, they may be synthesized according to the processes described, for example, in U.S. Pat. Nos. 6,225,198 or 5,990,479, in the publications cited therein and also in the following publications: Dabboussi B. O. et al., “(CdSe)ZnS core-shell quantum dots: synthesis and characterization of a size series of highly luminescent nanocrystallites”, Journal of Physical Chemistry B, vol. 101, 1997, pages 9463-9475, and Peng, Xiaogang et al., “Epitaxial growth of highly luminescent CdSe/CdS core/shell nanocrystals with photostability and electronic accessibility”, Journal of the American Chemical Society, vol. 119, No. 30, pages 7019-7029.

The variety of pigments that may be used in the present invention makes it possible to obtain a wide range of colors, and also particular optical effects such as metallic effects or interference effects.

The size of the pigment used in the composition according to the present invention is generally of between 10 nm and 200 μm, preferably between 20 nm and 80 μm, more preferentially between 30 nm and 50 μm and even more preferentially between 80 nm and 10 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants moreover contain at least one functional group that is compatible with or soluble in the continuous medium. In particular, 12-hydroxystearic acid esters in particular and C₈ to C₂₀ fatty acid esters of polyols such as glycerol or diglycerol are used, such as poly(12-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or else polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments that have been surface-treated beforehand, which are useful in the context of the invention, are pigments that have totally or partially undergone a surface treatment of chemical, electronic, electrochemical, mechanochemical or mechanical nature, with an organic agent such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol, lauric acid and derivatives thereof; anionic surfactants; lecithins; sodium, potassium, magnesium, iron, titanium, zinc or aluminum salts of fatty acids, for example aluminum stearate or laurate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by solvent evaporation, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight of the total weight of the surface-treated pigment, preferably from 0.5% to 30% by weight and even more preferentially from 1% to 20% by weight of the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminum dimyristate treatment, such as the MI surface treatment sold by Miyoshi;
a perfluoropolymethylisopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or mineral pigments in submicron-sized particulate form in the composition according to the invention.

The term “submicron” or “submicronic” means pigments having a particle size that has been micronized by a micronization method and having a mean particle size of less than a micrometer (μm), in particular between 0.1 and 0.9 μm, and preferably between 0.2 and 0.6 μm.

According to one embodiment, the dispersant and the pigment(s) are present in a weight ratio (dispersant:pigment) of between 1:4 and 4:1, particularly between 1.5:3.5 and 3.5:1 or better still between 1.75:3 and 3:1.

The dispersant(s) may therefore have a silicone backbone, such as silicone polyether and dispersants of aminosilicone type other than the aminosilicones as described previously. Among the suitable dispersants, mention may be made of:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones with carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of aminosilicone type other than the aminosilicones as described previously and are cationic.

Preferably, the pigment(s) is(are) chosen from mineral, mixed mineral-organic or organic pigments.

The composition may comprise one or more direct dyes.

In one variant of the invention, the pigment(s) according to the invention are organic pigments, preferentially organic pigments surface-treated with an organic agent chosen from silicone compounds. In another variant of the invention, the pigment(s) according to the invention are mineral pigments.

The term “direct dye” means natural and/or synthetic dyes, other than oxidation dyes. These are dyes that will spread superficially on the fiber.

They may be ionic or nonionic, preferably cationic or nonionic.

These direct dyes are chosen, for example, from neutral, acidic or cationic nitrobenzene direct dyes, neutral, acidic or cationic azo direct dyes, tetraazapentamethine dyes, neutral, acidic or cationic quinone and in particular anthraquinone dyes, azine direct dyes, triarylmethane direct dyes, azomethine direct dyes and natural direct dyes.

Examples of suitable direct dyes that may be mentioned include azo direct dyes; (poly)methine dyes such as cyanines, hemicyanines and styryl dyes; carbonyl dyes; azine dyes; nitro(hetero)aryl dyes; tri(hetero)arylmethane dyes; porphyrin dyes; phthalocyanine dyes, and natural direct dyes, alone or as mixtures.

Preferentially, the direct dye(s) contain at least one quaternized cationic chromophore or at least one chromophore bearing a quaternized or quaternizable cationic group.

According to a particular embodiment of the invention, the direct dyes comprise at least one quaternized cationic chromophore.

As direct dyes according to the invention, mention may be made of the following dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines such as azacarbocyanines, diazacarbocyanines, diazahemicyanines, hemicyanines, or tetraazacarbocyanines; diazines; diketopyrrolopyrroles; dioxazines; diphenylamines; diphenylmethanes; dithiazines; flavonoids such as flavanthrones and flavones; fluorindines; formazans; indamines; indanthrones; indigoids and pseudo-indigoids; indophenols; indoanilines; isoindolines; isoindolinones; isoviolanthrones; lactones; (poly)methines such as dimethines of stilbene or styryl type; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines, thioindigo; thiopyronines; triarylmethanes, or xanthenes.

For the cationic azo dyes, mention may be made particularly of those resulting from the cationic dyes described in Kirk-Othmer's Encyclopedia of Chemical Technology, “Dyes, Azo”, J. Wiley & Sons, updated on Apr. 19, 2010.

Among the azo dyes that may be used according to the invention, mention may be made of the cationic azo dyes described in patent applications WO 95/15144, WO 95/01772 and EP-714954.

According to a preferred embodiment of the invention, the direct dye(s) are chosen from cationic dyes known as “basic dyes”.

Among the azo dyes described in the Colour Index International 3rd edition, mention may be made notably of the following compounds:
Basic Red 22;
Basic Red 76;
Basic Yellow 57;
Basic Brown 16;
Basic Brown 17.

Among the cationic quinone dyes, those mentioned in the abovementioned Colour Index International are suitable for use and, among these, mention may be made, inter alia, of the following dyes:
Basic Blue 22;
Basic Blue 99.

Among the azine dyes that are suitable for use, mention may be made of those listed in the Colour Index International, for example the following dyes:
Basic Blue 17;
Basic Red 2.

Among the cationic triarylmethane dyes that may be used according to the invention, mention may be made, in addition to those listed in the Colour Index, of the following dyes:
Basic Green 1;
Basic Violet 3;
Basic Violet 14;
Basic Blue 7;
Basic Blue 26.

Mention may also be made of the cationic dyes described in U.S. Pat. No. 5,888,252, EP 1 133 975, WO 03/029 359, EP 860 636, WO 95/01772, WO 95/15144 and EP 714 954. Mention may also be made of those listed in the encyclopedia “The Chemistry of Synthetic Dyes” by K. Venkataraman, 1952, Academic Press, vol. 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons.

Preferably, the cationic direct dyes are chosen from those resulting from dyes of azo and hydrazono type.

According to a particular embodiment, the direct dyes are cationic azo dyes, described in EP 850 636, FR 2 788 433, EP 920 856, WO 99/48465, FR 2 757 385, EP 850 637, EP 918 053, WO 97/44004, FR 2 570 946, FR 2 285 851, DE 2 538 363, FR 2 189 006, FR 1 560 664, FR 1 540 423, FR 1 567 219, FR 1 516 943, FR 1 221 122, DE 4 220 388, DE 4 137 005, WO 01/66646, U.S. Pat. No. 5,708,151, WO 95/01772, WO 515 144, GB 1 195 386, U.S. Pat. Nos. 3,524,842, 5,879,413, EP 1 062 940, EP 1 133 976, GB 738 585, DE 2 527 638, FR 2 275 462, GB 1974-27645, Acta Histochem. (1978), 61(1), 48-52; Tsitologiya (1968), 10(3), 403-5; Zh. Obshch. Khim. (1970), 40(1), 195-202; Ann. Chim. (Rome) (1975), 65(5-6), 305-14; Journal of the Chinese Chemical Society (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dye(s) comprise a quaternary ammonium group; more preferentially, the cationic charge is endocyclic.

These cationic radicals are, for example, a cationic radical:
bearing an exocyclic (di/tri)(C₁-C₈)alkylammonium charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthooxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the hydrazono cationic dyes of formulae (X) and (XI) and the azo dyes of formulae (XII) and (XIII) below:

[Chem. 14]

Het⁺-C(Ra)═N—N(Rb)—Ar,Q⁻ (X)

[Chem. 15]

Het⁺—N═C(Rb)—Ar,Q⁻ (XI)

[Chem. 16]

Het⁺—N═N—Ar,Q⁻ (XII)

[Chem. 17]

Ar⁺—N═N—Ar″,Q⁻ (XIII)

in which formula (X) to (XIII):
Het⁺ represents a cationic heteroaryl radical, preferentially bearing an endocyclic cationic charge, such as imidazolium, indolium or pyridinium, which is optionally substituted, preferentially with at least one (C₁-C₈) alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group;
   or else the substituent Ra with a substituent of Het+ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q- represents an organic or mineral anionic counterion, such as a halide or an alkyl sulfate.

In particular, mention may be made of the azo and hydrazono direct dyes bearing an endocyclic cationic charge of formulae (X) to (XIII) as defined previously. More particularly, the cationic direct dyes bearing an endocyclic cationic charge described in patent applications WO 95/15144, WO 95/01772 and EP 714 954. Preferentially the following direct dyes:

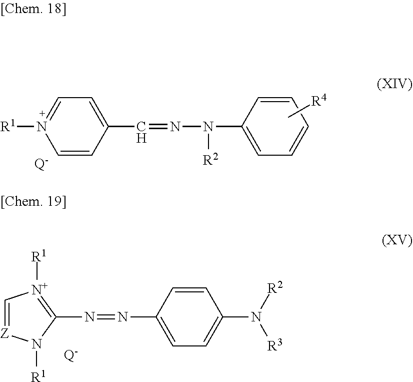

in which formulae (XIV) and (XV):
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH,
Q- is an anionic counterion as defined previously, in particular a halide, such as chloride, or an alkyl sulfate, such as methyl sulfate or mesityl.

Particularly, the dyes of formulae (XIV) and (XV) are chosen from Basic Red 51, Basic Yellow 87 and Basic Orange 31 or derivatives thereof:

According to a particular embodiment of the invention, the direct dyes are fluorescent, i.e. they contain at least one fluorescent chromophore as defined previously.

Fluorescent dyes that may be mentioned include the radicals resulting from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (in particular cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes and xanthenes.

Mention may also be made of the fluorescent dyes described in EP 1 133 975, WO 03/029 359, EP 860 636, WO 95/01772, WO 95/15144 and EP 714 954 and those listed in the encyclopedia “The Chemistry of Synthetic Dyes” by K. Venkataraman, 1952, Academic Press, vol. 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons, and in the handbook “A Guide to Fluorescent Probes and Labeling Technologies”, 10th Ed., Molecular Probes/Invitrogen—Oregon 2005, circulated on the Internet or in the preceding printed editions.

According to a preferred variant of the invention, the fluorescent dye(s) are cationic and comprise at least one quaternary ammonium radical, such as those of formula (XVI) below:

[Chem. 20]

W⁺—[C(R_{c})═C(R_{d})]_{m′}—Ar,Q⁻ (XVI)

in which formula (XVI):
W⁺ represents a cationic heterocyclic or heteroaryl group, particularly comprising a quaternary ammonium optionally substituted with one or more (C₁-C₈)alkyl groups, optionally substituted notably with one or more hydroxyl groups;
Ar representing an aryl group such as phenyl or naphthyl, optionally substituted preferentially with i) one or more halogen atoms such as chlorine or fluorine; ii) one or more (C₁-C₈)alkyl groups, preferably of C₁-C₄ such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyls, such as (di)hydroxyethylamino, vii) with one or more acylamino groups; viii) one or more heterocycloalkyl groups such as piperazinyl, piperidinyl or 5- or 6-membered heteroaryl such as pyrrolidinyl, pyridinyl and imidazolinyl;
m′ represents an integer between 1 and 4 inclusive; in particular, m is 1 or 2; more preferentially 1;
R_{c} and R_{d}, which may be identical or different, represent a hydrogen atom or an optionally substituted (C₁-C₈)alkyl group, preferentially of C₁-C₄, or alternatively R_{c} contiguous with W⁺ and/or R_{d} contiguous with Ar form, with the atoms that bear them, a (hetero)cycloalkyl; particularly, R_{c} is contiguous with W⁺ and they form a (hetero)cycloalkyl such as cyclohexyl;
Q- is an organic or mineral anionic counterion as defined previously.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions comprising these natural dyes and in particular henna-based poultices or extracts.

The composition may comprise one or more pigments present in a total content ranging from 0.05% to 30% by weight, preferably from 0.1% to 20% by weight and better still from 0.1% to 15% by weight relative to the total weight of the composition.

The composition may comprise one or more direct dyes present in a total content ranging from 0.001% to 10% by weight, preferably from 0.005% to 5% by weight relative to the total weight of the composition.

### Oils

The composition may comprise one or more oil(s).

Preferably, the composition comprises one or more oil(s) chosen from alkanes.

The term “oil” means a fatty substance that is liquid at room temperature (25° C.) and at atmospheric pressure (760 mmHg or 1.013×10⁵ Pa).

The oil may be volatile or nonvolatile.

The term “volatile oil” means an oil that can evaporate on contact with the skin in less than one hour, at room temperature and atmospheric pressure. The volatile oil is a cosmetic volatile oil, which is liquid at room temperature. More specifically, a volatile oil has an evaporation rate of between 0.01 and 200 mg/cm²/min, limits included (see protocol for measuring the evaporation rate indicated in the text below).

The term “nonvolatile oil” means an oil that remains on the skin or the keratin fiber at room temperature and atmospheric pressure. More specifically, a nonvolatile oil has an evaporation rate of strictly less than 0.01 mg/cm²/min (see protocol for measuring the evaporation rate indicated in the text below).

Preferably, the composition comprises one or more oil(s) chosen from C₆-C₁₆ alkanes and/or mixtures thereof.

As regards the C₆-C₁₆ alkanes, they may be linear or branched, and possibly cyclic.

Mention may notably be made of branched C₈-C₁₆ alkanes, such as C₈-C₁₆ isoalkanes (also known as isoparaffins), isododecane, isodecane or isohexadecane, and for example the oils sold under the Isopar or Permethyl trade names, and mixtures thereof.

Mention may also be made of linear alkanes, preferably of plant origin, comprising from 7 to 15 carbon atoms, in particular from 9 to 14 carbon atoms and more particularly from 11 to 13 carbon atoms.

As examples of linear alkanes that are suitable for use in the invention, mention may be made of n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and n-pentadecane (C₁₅), and mixtures thereof, and in particular the mixture of n-undecane (C₁₁) and n-tridecane (C₁₃) described in Example 1 of patent application WO 2008/155 059 by the company Cognis.

Mention may also be made of n-dodecane (C₁₂) and n-tetradecane (C₁₄) sold by Sasol under the references, respectively, Parafol 12-97 and Parafol 14-97, and also mixtures thereof.

As examples of alkanes that are suitable for use in the invention, mention may be made of the alkanes described in patent applications WO 2007/068 371 and WO 2008/155 059. These alkanes are obtained from fatty alcohols, which are themselves obtained from coconut kernel oil or palm oil.

According to a particular embodiment, the composition comprises isododecane. Such a compound is, for example, the isododecane sold under the reference Isododecane by Ineos.

Preferably, the composition according to the invention comprises one or more oils chosen from C₈-C₁₆ alkanes, more preferentially from isododecane, isohexadecane, tetradecane and/or mixtures thereof.

More preferentially, the composition comprises isododecane.

The composition according to the invention may comprise one or more oils present in a total amount of between 10% and 99% by weight, preferably between 20% and 95% by weight and better still between 30% and 90% by weight, relative to the total weight of the composition.

### Mass Ratio:

The composition according to the invention may comprise the alkoxysilane(s) of formula (VIII) and the amino silicone(s) in a mass ratio of alkoxysilane(s) of formula (VIII)/amino silicone(s) ranging from 95:5 to 5:95.

Preferably, the mass ratio of alkoxysilane(s) of formula (VIII)/amino silicone(s) ranges from 90:10 to 10:90, preferentially from 80:20 to 20:80, more preferentially from 70:30 to 30:70.

According to a preferred embodiment, the mass ratio of APTES/amino silicone(s) ranges from 90:10 to 10:90, preferentially from 80:20 to 20:80, more preferentially from 70:30 to 30:70.

### Additives:

The composition may also contain any adjuvant or additive usually used.

Among the additives that may be contained in the composition, mention may be made of reducing agents, thickeners, softeners, antifoams, moisturizers, UV-screening agents, peptizers, solubilizers, fragrances, anionic, cationic, nonionic or amphoteric surfactants, proteins, vitamins, polymers, preserving agents, waxes and mixtures thereof.

The composition according to the invention may notably be in the form of a suspension, a dispersion or a gel, in the form of a cream, a mousse, a stick, a dispersion of vesicles, notably of ionic or nonionic lipids, a two-phase or multi-phase lotion, an anhydrous liquid or an anhydrous gel.

In a preferred embodiment, the cosmetic composition according to the invention is an anhydrous liquid or an anhydrous gel.

A person skilled in the art may select the appropriate presentation form, and also the method for preparing it, on the basis of his or her general knowledge, taking into account firstly the nature of the constituents used, notably their solubility in the support, and secondly the intended application of the composition.

According to a preferred embodiment, the anhydrous composition according to the invention comprises at least one amino silicone of formula (I) as described previously, at least one alkoxysilane of formula (VIIIa) as described previously and at least one pigment.

According to a more preferred embodiment, the anhydrous composition according to the invention comprises at least one amino silicone of formula (III) as described previously, 3-aminopropyltriethoxysilane (APTES) and at least one pigment.

### Process for Treating Keratin Fibers

The present invention also relates to a process for the cosmetic treating of, in particular for dyeing, human keratin fibers such as the hair, in which the composition as described previously is applied to said fibers.

Preferably, the anhydrous composition according to the invention is a composition for dyeing human keratin fibers such as the hair.

The composition according to the invention may be used on wet or dry keratin fibers, and also on any type of fair or dark, natural or dyed, permanent-waved, bleached or relaxed fibers.

According to a particular embodiment of the process of the invention, the fibers are washed before applying the composition described above.

The application of the dye composition to the keratin fibers may be performed by any conventional means, in particular using a comb, a fine brush, a coarse brush or with the fingers.

The dyeing process, i.e. application of the dye composition to the keratin fibers, is generally performed at room temperature (between 15 and 25° C.).

The process according to the invention may comprise a step of applying heat to the keratin fibers using a heating tool.

The heat application step of the process of the invention may be performed using a hood, a hairdryer, a straightening or curling iron, a Climazon, etc.

Preferably, the heat application step of the process of the invention is performed using a hairdryer and/or a straightening iron, more preferentially using a straightening iron.

When the process of the invention involves a step of applying heat to the keratin fibers, the step of applying heat to the keratin fibers takes place after the application of the composition to the keratin fibers.

During the step of applying heat to the keratin fibers, a mechanical action may be exerted on the locks, such as combing, brushing or running the fingers through.

When the step of applying heat to the keratin fibers is performed using a hood or a hairdryer, the temperature is preferably between 30° C. and 110° C., preferentially between 50° C. and 90° C.

When the step of applying heat to the keratin fibers is performed using a straightening iron, the temperature is preferably between 110° C. and 220° C., preferably between 140° C. and 200° C.

In a particular variant, the process of the invention involves a step (b1) of applying heat using a hood, a hairdryer or a Climazon, preferably a hairdryer, and a step (b2) of applying heat using a straightening or curling iron, preferably a straightening iron.

Step (b1) may be performed before step (b2).

During step (b1), also referred to as the drying step, the fibers may be dried, for example at a temperature above or equal to 30° C. According to a particular embodiment, this temperature is above 40° C. According to a particular embodiment, this temperature is above 45° C. and below 110° C.

Preferably, if the fibers are dried, they are dried, in addition to a supply of heat, with a flow of air. This flow of air during drying makes it possible to improve the strand separation of the coating.

During drying, a mechanical action may be exerted on the locks, such as combing, brushing or running the fingers through.

During step (b2), the passage of the straightening or curling iron, preferably the straightening iron, may be performed at a temperature ranging from 110° C. to 220° C., preferably between 140° C. and 200° C.

The present invention also relates to a process for treating human keratin fibers such as the hair, comprising the following steps:

a) applying to the keratin fibers an anhydrous composition as defined above, and
b) applying to the dyed keratin fibers a makeup-removing composition comprising at least one hydrocarbon-based oil.

The present invention also relates to a kit for treating keratin fibers such as the hair, comprising an anhydrous composition as defined above, and a makeup-removing composition comprising at least one hydrocarbon-based oil.

Thus, the process according to the invention may comprise a step of applying a makeup-removing composition to dyed keratin fibers such as the hair. This step may take place following the dyeing of the keratin fibers with the dye composition according to the invention, i.e. anhydrous composition, after the optional step of applying heat to the keratin fibers, or after a defined time, i.e. days or weeks, after the application of the dye composition to the keratin fibers and the optional step of applying heat to the keratin fibers.

The makeup-removing composition may correspond to a makeup-removing composition conventionally used in cosmetics. Preferably, the makeup-removing composition comprises at least one hydrocarbon-based oil.

Preferably, the hydrocarbon-based oil(s) are chosen from synthetic esters of formula R₁COOR₂ in which R₁ represents a fatty acid residue including from 8 to 29 carbon atoms, and R₂ represents a branched or unbranched hydrocarbon-based chain containing from 3 to 30 carbon atoms, and mixtures thereof, more preferentially from isopropyl myristate, isononyl isononanoate and/or mixtures thereof.

The application of the dye composition to the keratin fibers such as the hair may be performed before the application of a makeup-removing composition.

The step of applying heat to the keratin fibers may be performed after the application of the dye composition and before the application of the makeup-removing composition to the keratin fibers.

The makeup-removing composition may be applied to keratin fibers dyed with the dye composition according to the invention as defined previously.

The application of the makeup-removing composition may be performed on dry dyed keratin fibers or wet dyed keratin fibers and also on all types of fibers.

The makeup-removing process is generally performed at room temperature (between 15° C. and 25° C.).

The application of the makeup-removing composition may be performed immediately after the application of the dye composition (i.e. a few minutes to a few hours after the application of the dye composition), or in the days or weeks following the application of the dye composition.

The makeup-removing composition may be applied with the aid of any suitable support, which is notably capable of absorbing it, for example a fibrous makeup-removing disk, for example a woven or nonwoven fabric, cotton wool, a flocked film, a sponge, a wipe, or a twisted or injection-molded mascara application brush.

The makeup-removing composition may be contained in a container and taken up gradually each time makeup is removed. As a variant, the makeup-removing composition impregnates the support used for makeup removal, the support possibly being packaged, in this case, for example in leaktight packaging.

After the makeup-removing composition has been used, it is possible for the keratin materials to be not rinsed. As a variant, they may be rinsed. The rinsing may be performed, for example, with running water, without addition of a soap.

The present invention also relates to the use of the composition as defined previously, for the cosmetic treatment, in particular the dyeing, of keratin fibers, in particular human keratin fibers such as the hair.

The present invention will now be described more specifically by means of examples, which do not in any way limit the scope of the invention. However, the examples make it possible to support specific features, variants and preferred embodiments of the invention.

### EXAMPLE

### Example 1

Dye composition: Compositions (g/100 g)

TABLES 1

| | A | B | C | D |
|---|---|---|---|---|
| Composition | (invention) | (comparative) | (invention) | (comparative) |
| Amodimethicone¹⁾ | 8 | 8 | — | — |
| Amodimethicone²⁾ | — | — | 8 | 8 |
| Aminopropyltriethoxysilane ⁽³⁾ | 3 | 3 | 3 | 3 |
| Red 7 lake (and) | 5 | 5 | 5 | 5 |
| Isopropyl titanium | | | | |
| triisostearate (and) | | | | |
| Triethoxysilylethyl | | | | |
| polydimethylsiloxyethyl) | | | | |
| Cetyl PEG/PPG- | — | 1 | — | 1 |
| 10/1 dimethicone⁽⁴⁾ | | | | |
| Isododecane | qs 100 | 12 | qs 100 | 12 |
| Water | — | qs 100 | — | qs 100 |

¹⁾sold under the trade name KF857 by the company Shin-Etsu,
²⁾sold under the trade name KF862 by the company Shin-Etsu,
⁽³⁾ sold under the trade name Silsoft A-1100 by the company Momentive Performance Materials,
⁽⁴⁾sold under the trade name Abil EM 90 by the company Evonik Goldschmidt.

(1) sold under the trade name KF857 by the company Shin-Etsu,
(2) sold under the trade name KF862 by the company Shin-Etsu,
(3) sold under the trade name Silsoft A-1100 by the company Momentive Performance Materials,
(4) sold under the trade name Abil EM 90 by the company Evonik Goldschmidt.

Compositions A and C according to the invention are anhydrous compositions. Comparative compositions B and D are in the form of an O/W emulsion.

Protocol:

Compositions A and C according to the invention and comparative compositions B and D are applied to locks of natural hair containing 90% white hairs, at a rate of 0.5 g of composition per gram of lock.

The locks of hair are then combed and dried with a hairdryer.

The locks of hair thus dyed are then subjected to a test of several repeated shampoo washes so as to evaluate the resistance (persistence) of the coloring obtained with respect to shampoo washing.

Shampoo Wash Protocol:

The locks are washed with a standard shampoo (Gamier Ultra Doux) respectively at T=24 hours after the application of the dye composition to the keratin fibers.

The locks of hair are then rinsed, combed and dried with a hairdryer.

The next shampoo wash is then performed on the locks thus dried.

Results:

The persistence of the color of the locks was evaluated in the CIE L*a*b* system, using a Minolta Spectrophotometer CM3600D colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone five shampoo washes according to the protocol described above. The lower the value of ΔE, the more persistent the color with respect to shampoo washing.

TABLES 2

| | Number of | | | | |
|---|---|---|---|---|---|
| | shampoo | | | | |
| Compositions | washes | L* | a* | b* | ΔE |
| Composition A | 0 | 32.83 | 36.97 | 11.4 | — |
| | 5 | 56.52 | 14.16 | 9.41 | 32.95 |
| Composition B | 0 | 29.69 | 41.36 | 20.26 | — |
| | 5 | 54.47 | 15.94 | 7.75 | 37.64 |
| Composition C | 0 | 35.12 | 42.19 | 15.4 | — |
| | 5 | 48.82 | 19.06 | 8.43 | 27.77 |
| Composition D | 0 | 31.61 | 40.75 | 17.54 | — |
| | 5 | 51.79 | 15.06 | 7.91 | 34.06 |

The hair treated with the compositions according to the invention has a natural feel and the strands of hair may be separated with the fingers or by using a comb and/or a brush.

The locks of hair dyed with the anhydrous compositions A and C according to the invention and washed with five shampoo washes have ΔE values that are lower than those of the locks of hair dyed with the comparative compositions B and D in O/W emulsion form.

Thus, the colored coating of the keratin fibers obtained with the anhydrous compositions A and C according to the invention shows good persistence with respect to shampoo washing. Specifically, the locks of hair dyed with the anhydrous compositions A and C according to the invention and washed with five shampoo washes have better persistence of the color than the locks of hair dyed with the comparative compositions B and D in O/W emulsion form.

### Example 2

Dye composition: Compositions (g/100 g)

TABLES 3

| | E | F | G | H |
|---|---|---|---|---|
| Composition | (invention) | (comparative) | (invention) | (comparative) |
| Amodimethicone¹⁾ | 8 | 8 | — | — |
| Amodimethicone²⁾ | — | — | 8 | 8 |
| Aminopropyltriethoxysilane ⁽³⁾ | 3 | 3 | 3 | 3 |
| Pigment green 7 | 5 | 5 | 5 | 5 |
| (CI 74260) | | | | |
| Cetyl PEG/PPG- | — | 1 | — | 1 |
| 10/1 dimethicone⁽⁴⁾ | | | | |
| Isododecane | qs 100 | 12 | qs 100 | 12 |
| Water | — | qs 100 | — | qs 100 |

¹⁾sold under the trade name KF857 by the company Shin-Etsu,
²⁾sold under the trade name KF862 by the company Shin-Etsu,
⁽³⁾ sold under the trade name Solsoft A-1100 by the company Momentive Performance Materials,
⁽⁴⁾sold under the trade name Abil EM 90 by the company Evonik Goldschmidt.

(1) sold under the trade name KF857 by the company Shin-Etsu,
(2) sold under the trade name KF862 by the company Shin-Etsu,
(3) sold under the trade name Silsoft A-1100 by the company Momentive Performance Materials,
(4) sold under the trade name Abil EM 90 by the company Evonik Goldschmidt.

Compositions E and G according to the invention are anhydrous compositions. Comparative compositions F and H are in the form of an O/W emulsion.

Protocol:

Compositions E and G according to the invention and comparative compositions F and H are applied to locks of natural hair containing 90% white hairs, at a rate of 0.5 g of composition per gram of lock.

The locks of hair are then combed and dried with a hairdryer.

The locks of hair thus dyed are then subjected to a test of several repeated shampoo washes so as to evaluate the resistance (persistence) of the coloring obtained with respect to shampoo washing.

Shampoo Wash Protocol:

The locks are washed with a standard shampoo (Gamier Ultra Doux) respectively at T=24 hours after the application of the dye composition to the keratin fibers.

The locks of hair are then rinsed, combed and dried with a hairdryer.

The next shampoo wash is then performed on the locks thus dried.

Results:

The persistence of the color of the locks was evaluated in the CIE L*a*b* system, using a Minolta Spectrophotometer CM3600D colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone five shampoo washes according to the protocol described above. The lower the value of ΔE, the more persistent the color with respect to shampoo washing.

TABLES 4

| | Number of | | | | |
|---|---|---|---|---|---|
| | shampoo | | | | |
| Compositions | washes | L* | a* | b* | ΔE |
| Composition E | 0 | 31.5 | −28.28 | 4.2 | — |
| | 5 | 57.6 | −26.66 | 10.79 | 26.97 |
| Composition F | 0 | 26.3 | −28.63 | 3.67 | — |
| | 5 | 61.98 | −18.55 | 14.5 | 38.63 |
| Composition G | 0 | 35.69 | −31.58 | 5.64 | — |
| | 5 | 53.21 | −19.06 | 10.86 | 22.16 |
| Composition H | 0 | 31.54 | −28.46 | 4.8 | — |
| | 5 | 59.4 | −17.27 | 13.38 | 31.23 |

The hair treated with the compositions according to the invention has a natural feel and the strands of hair may be separated with the fingers or by using a comb and/or a brush.

The locks of hair dyed with the anhydrous compositions E and G according to the invention and washed with five shampoo washes have ΔE values that are lower than those of the locks of hair dyed with the comparative compositions F and H in O/W emulsion form.

Thus, the colored coating of the keratin fibers obtained with the anhydrous compositions E and G according to the invention shows good persistence with respect to shampoo washing. Specifically, the locks of hair dyed with the anhydrous compositions E and G according to the invention and washed with five shampoo washes have better persistence of the color than the locks of hair dyed with the comparative compositions F and H in 0/W emulsion form.

### Example 3

Dye composition: Compositions (g/100 g)

TABLES 5

| | I | J | K |
|---|---|---|---|
| Composition | (invention) | (comparative) | (comparative) |
| Amodimethicone¹⁾ | 8 | 8 | 8 |
| Aminopropyltriethoxysilane ⁽²⁾ | 3 | — | — |
| Hexamethyldisiloxane ⁽³⁾ | — | 3 | — |
| Red 7 lake (and) Isopropyl | 5 | 5 | 5 |
| titanium triisostearate (and) | | | |
| Triethoxysilylethyl | | | |
| polydimethylsiloxyethyl) | | | |
| Isododecane | qs 100 | qs 100 | qs 100 |

¹⁾sold under the trade name KF857 by the company Shin-Etsu,
⁽²⁾ sold under the trade name Silsoft A-1100 by the company Momentive Performance Materials,
⁽³⁾ sold under the trade name Xiameter PMX-200 silicone fluid .65 cst by the company Dow Corning.

(1) sold under the trade name KF857 by the company Shin-Etsu,
(2) sold under the trade name Silsoft A-1100 by the company Momentive Performance Materials,
(3) sold under the trade name Xiameter PMX-200 silicone fluid 0.65 cst by the company Dow Corning.

Protocol:

Composition I according to the invention and comparative compositions J and K are applied to locks of natural hair containing 90% white hairs, at a rate of 0.5 g of composition per gram of lock.

The locks of hair are then combed and dried with a hairdryer.

The locks of hair thus dyed are then subjected to a test of several repeated shampoo washes so as to evaluate the resistance (persistence) of the coloring obtained with respect to shampoo washing.

Shampoo Wash Protocol:

The locks are washed with a standard shampoo (Gamier Ultra Doux) respectively at T=24 hours after the application of the dye composition to the keratin fibers.

The locks of hair are then rinsed, combed and dried with a hairdryer.

The next shampoo wash is then performed on the locks thus dried.

Results:

The persistence of the color of the locks was evaluated in the CIE L*a*b* system, using a Minolta Spectrophotometer CM3600D colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone three shampoo washes according to the protocol described above. The lower the value of ΔE, the more persistent the color with respect to shampoo washing.

TABLES 6

| | Number of | | | | |
|---|---|---|---|---|---|
| | shampoo | | | | |
| Compositions | washes | L* | a* | b* | ΔE |
| Composition I | 0 | 32.72 | 42.75 | 15.76 | — |
| | 3 | 45.14 | 26.41 | 7.05 | 22.30 |
| Composition J | 0 | 32.24 | 45.21 | 18.56 | — |
| | 3 | 50.69 | 20.75 | 6.16 | 33.05 |
| Composition K | 0 | 32.36 | 41.51 | 16.91 | — |
| | 3 | 51.43 | 16.92 | 4.71 | 33.42 |

The locks of hair dyed with the anhydrous composition I according to the invention and washed with three shampoo washes have ΔE values that are lower than those of the locks of hair dyed with the comparative compositions J and K.

Thus, the colored coating of the keratin fibers obtained with the anhydrous composition I according to the invention shows good persistence with respect to shampoo washing. Specifically, the locks of hair dyed with the anhydrous composition I according to the invention and washed with three shampoo washes have better persistence of the color than the locks of hair dyed with the comparative compositions J and K.

Make-up removing composition: (g/100 g)

TABLES 7

| Composition | L |
|---|---|
| Polysorbate 21 | 30 |
| Isopropyl myristate | 30 |
| Isononyl isononanoate | 30 |
| Ethanol | 3 |
| Caprylyl glycol | 0.5 |
| Preservative | Qs |
| Water | qs 100 |

Protocol:

The make-up removing composition L is applied to locks of hair dyed with compositions I, J and K, at a rate of 1 gr of composition per gram of lock. The locks are then kneaded from top to tip for 5 min before rinsing.

This manipulation was repeated a second time before drying the locks with a hairdryer.

Results:

The efficacy of the make-up removing composition was evaluated in the CIE L*a*b* system, using a Minolta Spectrophotometer CM3600D colorimeter (illuminant D65, angle 10°, specular component included) as described above.

The efficacy of the make-up removing composition is evaluated by the color difference ΔE between the dyed locks before applying the make-up removing composition L, then after applying the make-up removing composition L according to the protocol described above. The higher the value of ΔE, the more color is removed from the dyed locks.

TABLES 8

| Compositions | Step | L* | a* | b* | ΔE |
|---|---|---|---|---|---|
| Composition I | after dyeing | 32.72 | 42.75 | 15.76 | — |
| | after applying | 58.30 | 7.50 | 9.76 | 43.96 |
| | composition L | | | | |
| Composition J | after dyeing | 32.24 | 45.21 | 18.56 | — |
| | after applying | 57.99 | 9.56 | 9.81 | 44.84 |
| | composition L | | | | |
| Composition K | after dyeing | 32.36 | 41.51 | 16.91 | — |
| | after applying | 58.44 | 7.44 | 9.85 | 43.48 |
| | composition L | | | | |

The application of the makeup-removing composition L on the locks of hair dyed with composition I, J or K leads to high values of ΔE, therefore to a significant loss of color on the locks on hair dyed.

1-17. (canceled)

18. A composition comprising:
a) at least one amino silicone,
b) at least one alkoxysilane chosen from the compounds of formula (VIII) below, oligomers thereof and/or mixtures thereof:
R¹ₓSi(OR²)₍₄₋ₓ₎ (VIII),
wherein in formula (VIII):
R¹ represents an alkoxy group containing from 1 to 10 carbon atoms; or a linear or branched, saturated or unsaturated, cyclic or acyclic C₁ to C₂₂ hydrocarbon-based radical; which may be substituted with at least one group chosen from a hydroxyl group (OH); a thiol group; an amino group NH₂; an alkylamino group NHR in which R denotes a linear or branched alkyl radical containing from 1 to 20 carbon atoms, or an alkoxy group containing from 1 to 10 carbon atoms; a cycloalkyl containing from 3 to 40 carbon atoms; an aryl containing from 6 to 30 carbon atoms; R¹ possibly being interrupted with at least one heteroatom chosen from O, S, NH or a carbonyl group (CO);
R² represents a hydrogen atom or an alkyl group containing from 1 to 20 carbon atoms;
x denotes an integer ranging from 1 to 3; and
c) at least one coloring agent chosen from pigments, direct dyes, or mixtures thereof.

19. The composition of claim 18, wherein the at least one amino silicone is chosen from silicones of formula (I) below: 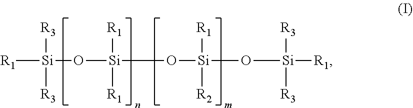
wherein in formula (I):
which may be identical or different, represents a hydrogen atom, a phenyl group, a hydroxyl group, a C₁-C₈ alkyl group, or a C₁-C₆ alkoxy group;
R₂ represents a monovalent radical of formula —CqH2qL, wherein q is a number ranging from 2 to 8 and L is an optionally quaternized amino group chosen from the following groups:
—N(R″)₂,—N+(R″)₃A⁻,—NR″-Q-N(R″)₂, or —NR″-Q-N⁺(R″)₃A⁻,
wherein R″, which may be identical or different, represents a hydrogen atom, a phenyl group, a benzyl group, or a saturated monovalent hydrocarbon-based radical; Q denotes a linear or branched group of formula CrH₂r, r being an integer ranging from 2 to 6; and A⁻ represents a cosmetically acceptable anion;
R₃, which may be identical or different, represents a C₁-C₈ alkyl group, or a monovalent radical of formula —CqH2qL, wherein q is a number ranging from 2 to 8 and L is an optionally quaternized amine group chosen from the following groups:
—N(R″)₂;—N+(R″)₃A⁻;—NR″-Q-N(R″)₂ and —NR″-Q-N⁺(R″)₃A⁻,
wherein R″, which may be identical or different, represents a hydrogen atom, a phenyl group, a benzyl group, or a saturated monovalent hydrocarbon-based radical; Q denotes a linear or branched group of formula CrH₂r, r being an integer ranging from 2 to 6; and A⁻ represents a cosmetically acceptable anion; and
m and n are numbers such that the sum (n+m) ranges from 1 to 2000, m denoting a number from 0 to 1999, and n denoting a number from 1 to 2000.

20. The composition of claim 18, wherein the at least one amino silicone is chosen from the silicones of formula (II) below: 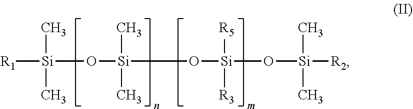
wherein in formula (II):
m and n are numbers such that the sum (m+n) ranges from 1 to 1000, n denoting a number between 0 and 999, and m denoting a number from 1 to 1000;
R₁ and R₂, which may be identical or different, represent a C₁-C₈ alkyl group, a hydroxyl group, or a C₁ to C₆ alkoxy group;
R₃ represents a monovalent radical of formula —CqH2qL, wherein q is a number ranging from 2 to 8 and L is an amino group chosen from the following groups:
—N(R₄)₂;
—N(R₄)—CH₂—CH₂—N(R₄)₂;
wherein R₄ represents a hydrogen atom, a phenyl group, a benzyl group, or a saturated monovalent hydrocarbon-based radical; and
R₅ represents a hydroxyl group, a C₁-C₈ alkyl group, or a C₁-C₋₆ alkoxy group.

21. The composition of claim 18, wherein the at least one amino silicone is chosen from the silicones of formula (III) below: 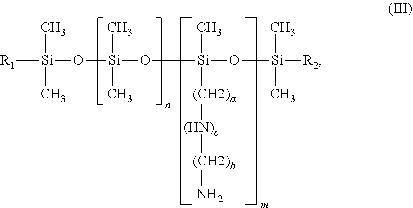
wherein in formula (III):
m and n are numbers such that the sum (m+n) ranges from 1 to 1000, n denoting a number between 0 and 999, and m denoting a number from 1 to 1000;
R₁ and R₂, which may be identical or different, represent a C₁ to C₄ alkoxy group;
a denotes an integer ranging from 0 to 6;
b denotes an integer ranging from 1 to 6; and
c denotes an integer equal to 0 or 1.

22. The composition of claim 18, wherein the at least on amino silicone is present in a total amount ranging from 0.1% to 40% by weight, relative to the total weight of the composition.

23. The composition of claim 18, wherein the at least one amino silicone is present in a total amount ranging from 1% to 20% by weight, relative to the total weight of the composition.

24. The composition of claim 18, wherein the at least one alkoxysilane is chosen from compounds of formulae (VIIIa) and (VIIIb) below, alone or as a mixture: 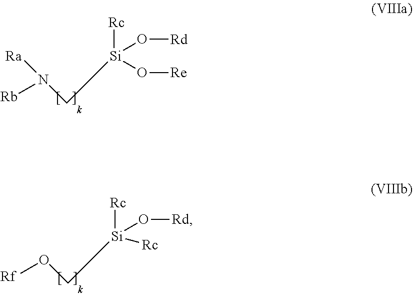
wherein in formulae (VIIIa) and (VIIIb):
Ra and Rb, which may be identical or different, represent a hydrogen atom, an alkyl group containing from 1 to 20 carbon atoms, a cycloalkyl group containing from 3 to 20 carbon atoms, an aryl group containing from 6 to 12 carbon atoms, an aminoalkyl group containing from 1 to 20 carbon atoms, a hydroxyalkyl group containing from 1 to 20 carbon atoms, an alkoxy group containing from 1 to 10 carbon atoms, or an alkylcarbonyl group containing from 1 to 10 carbon atoms;
Rc represents a hydrogen atom, an alkyl group containing from 1 to 20 carbon atoms, an alkoxy group containing from 1 to 10 carbon atoms, or an alkylaryl group containing from 7 to 12 carbon atoms;
Rd and Re, which may be identical or different, represent an alkyl group containing from 1 to 20 carbon atoms;
k denotes an integer ranging from 0 to 20; and
Rf represents a hydrogen atom, an alkyl group containing from 1 to 20 carbon atoms, or a group of formula (IX) below: 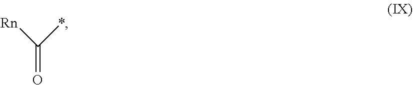
wherein Rn represents a hydroxyl group or an alkyl group containing from 1 to 20 carbon atoms.

25. The composition of claim 18, wherein the at least one alkoxysilane is chosen from compounds of formula (VIIIa) below: 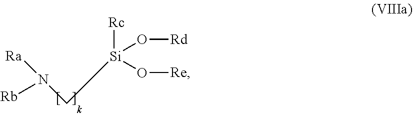
wherein in formula (VIIIa):
Ra and Rb, which are identical, represent a hydrogen atom;
Rc represents an alkyl group containing from 1 to 20 carbon atoms, or an alkoxy group containing from 1 to 10 carbon atoms;
Rd and Re, which may be identical or different, represent an alkyl group containing from 1 to 20 carbon atoms; and
k denotes an integer ranging from 0 to 20.

26. The composition of claim 18, wherein the at least one alkoxysilane chosen from compounds of formula (VIII) is chosen from 3-aminopropyltriethoxysilane (APTES), 3-aminopropylmethyldiethoxysilane (APMDES), N-cyclohexylaminomethyltriethoxysilane, tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane, or mixtures thereof.

27. The composition of claim 26, wherein the at least one alkoxysilane chosen from compounds of formula (VIII) comprises 3-aminopropyltriethoxysilane (APTES).

28. The composition of claim 18, wherein the at least one alkoxysilane chosen from compounds of formula (VIII) is present in a total amount ranging from 0.1% to 30% by weight, relative to the total weight of the composition.

29. The composition of claim 18, further comprising at least one oil chosen from C₈-C₁₆ alkanes.

30. The composition of claim 29, wherein the at least one oil is chosen from isododecane, isohexadecane, tetradecane, or mixtures thereof.

31. The composition of claim 18, wherein the composition further comprises at least one pigment present in a total content ranging from 0.05% to 30% by weight, relative to the total weight of the composition.

32. The composition of claim 18, further comprising a water content of less than 3% by weight, relative to the total weight of the composition.

33. The composition of claim 18, wherein the composition is free of water.

34. The composition of claim 18, wherein the mass ratio of the at least one alkoxysilane chosen from of formula (VIII) to the at least one amino silicone ranges from 95:5 to 5:95.

35. A process for dyeing human keratin fibers comprising applying to the keratin fibers a first composition comprising:
a) at least one amino silicone,
b) at least one alkoxysilane chosen from the compounds of formula (VIII) below, oligomers thereof and/or mixtures thereof:
R¹ₓSi(OR²)₍₄₋ₓ₎ (VIII),
wherein in formula (VIII):
R¹ represents alkoxy group containing from 1 to 10 carbon atoms, or a linear or branched, saturated or unsaturated, cyclic or acyclic C₁ to C₂₂ hydrocarbon-based radical, which may be substituted with at least one group chosen from a hydroxyl group (OH); a thiol group; an amino group NH₂; an alkylamino group NHR in which R denotes a linear or branched alkyl radical containing from 1 to 20 carbon atoms, or an alkoxy group containing from 1 to 10 carbon atoms; a cycloalkyl containing from 3 to 40 carbon atoms; an aryl containing from 6 to 30 carbon atoms; R¹ possibly being interrupted with at least one heteroatom chosen from 0, S, NH or a carbonyl group (CO);
R² represents a hydrogen atom or an alkyl group containing from 1 to 20 carbon atoms;
x denotes an integer ranging from 1 to 3; and
c) at least one colouring agent chosen from pigments, direct dyes, or mixtures thereof.

36. The process of claim 35, further comprising:
after the application of the first composition, applying to the keratin fibers a makeup-removing composition comprising at least one hydrocarbon-based oil.

37. A kit for treating keratin fibers, comprising:
an anhydrous composition; and
a makeup-removing composition comprising at least one hydrocarbon-based oil;
wherein the anhydrous composition comprises:
a) at least one amino silicone,
b) at least one alkoxysilane chosen from the compounds of formula (VIII) below, oligomers thereof and/or mixtures thereof:
R¹ₓSi(OR²)₍₄₋ₓ₎ (VIII),
wherein in formula (VIII):
R¹ represents alkoxy group containing from 1 to 10 carbon atoms, or a linear or branched, saturated or unsaturated, cyclic or acyclic C₁ to C₂₂ hydrocarbon-based radical, which may be substituted with at least one group chosen from a hydroxyl group (OH); a thiol group; an amino group NH₂; an alkylamino group NHR in which R denotes a linear or branched alkyl radical containing from 1 to 20 carbon atoms, or an alkoxy group containing from 1 to 10 carbon atoms; a cycloalkyl containing from 3 to 40 carbon atoms; an aryl containing from 6 to 30 carbon atoms; R¹ possibly being interrupted with at least one heteroatom chosen from 0, S, NH or a carbonyl group (CO);
R² represents a hydrogen atom or an alkyl group containing from 1 to 20 carbon atoms;
x denotes an integer ranging from 1 to 3; and
c) at least one colouring agent chosen from pigments, direct dyes, or mixtures thereof.
